(19) United States
(12) Patent Application Publication
(10) Pub. No.: US 2026/0204886 A1
(43) Pub. Date: Jul. 16, 2026

(54) CABLE PROTECTION APPARATUS, CABLE PROTECTION METHOD, AND PARTICLE BEAM TREATMENT SYSTEM

(71) Applicants: KABUSHIKI KAISHA TOSHIBA, Kawasaki-shi (JP); TOSHIBA ENERGY SYSTEMS & SOLUTIONS CORPORATION, Kawasaki-shi (JP)

(72) Inventors: Yuki ANZAI, Yokohama Kanagawa (JP); Yasuhiro YUGUCHI, Yokohama Kanagawa (JP); Kazuhito TOMITA, Yokohama Kanagawa (JP)

(73) Assignees: KABUSHIKI KAISHA TOSHIBA, Kawasaki-shi (JP); TOSHIBA ENERGY SYSTEMS & SOLUTIONS CORPORATION, Kawasaki-shi (JP)

(21) Appl. No.: 19/563,863

(22) Filed: Mar. 11, 2026

Related U.S. Application Data
(63) Continuation of application No. PCT/JP2024/043777, filed on Dec. 11, 2024.

(30) Foreign Application Priority Data
Apr. 1, 2024 (JP) 2024-058440

(51) Int. Cl. H02G 11/02; A61N 5/10

(52) U.S. Cl. CPC H02G 11/02; A61N 5/1081

(57) ABSTRACT

According to one embodiment, a cable protection apparatus includes: a rotating gantry configured to rotate about a horizontal axis oriented in a horizontal direction; at least one cable group in which a plurality of cables are bundled, each of the plurality of cables having one end connected to the rotating gantry and another end connected to a stationary apparatus; a spool provided on the rotating gantry and configured to wind or pay out the cable group; a cable straightening apparatus provided in a stationary state at a position below the spool and configured to straighten the cable group hanging down from the spool; and a flexible outer shell that surrounds at least a part of the cable group in a longitudinal direction of the cable group, at least a part of the outer shell being slidable along the longitudinal direction of the cable group.

### CROSS-REFERENCE TO RELATED APPLICATIONS

This application is a Continuation Application of No. PCT/JP2024/043777, filed on December 11, 2024, and the PCT application is based upon and claims the benefit of priority from Japanese Patent Applications No. 2024-058440, filed on April 1, 2024, the entire contents of which are incorporated herein by reference.

### TECHNICAL FIELD

Embodiments of the present invention relate to cable protection technology.

### BACKGROUND

In a rotating gantry of a particle beam treatment system, a large number of cables are connected to the rotating gantry, and thus, a spool is mounted thereon. The spool winds and pays out the cables, thereby maintaining the cable connection between equipment inside the rotating gantry and the outside regardless of the rotational angle of the rotating gantry.

When the number of cables to be wound or paid out increases, an irregular winding state may occur. To address this, techniques for preventing an irregular winding state of the cables are known.

For example, a plurality of cables are separated into lanes by brim disks, and a cable straightening apparatus is provided to straighten the cables along the lanes. The cable straightening apparatus includes components such as wires, plates, and rotating bodies. A monitoring apparatus provided with predetermined sensors prevents occurrence of an irregular winding state of the cables by monitoring the cables. However, even when cable straightening is performed by the cable straightening apparatus, the cables may hang down from the spool and a cable having a smaller diameter may become caught between the plurality of cables before being straightened by the cable straightening apparatus, thereby causing friction and causing the cables to deviate from its lane. The plurality of cables may become entangled within the lane. With these events, damage to or breakage of the cables may occur. The spool includes a penetration portion through which the cables extend from outside to inside. However, a cable led out through the penetration portion to the outside is subjected to a load resulting from the self-weight of the paid-out portion of the cable and may be damaged before reaching its service life.

Another known technique is to suspend a cable from a winding drum by its own weight for a required winding length. In such a case, increasing the axial length of the drum allows each cable to be stably wound and paid out without being entangled with other cables. However, in order to stably wind and pay out each cable without entanglement, it is necessary to increase the axial length of the winding drum, which consequently increases the overall length of the rotating gantry.

Still another known technique is to accommodate cables in a cable carrier and to wind and pay out the cables integrally with the cable carrier. The cables inside the cable carrier are restrained by clamps or binding straps to prevent irregular winding. In this technique, a supporter is known to support the cables in a multilayered manner. However, the supporter is any one of: a frame formed with a window through which the cables pass; a bar for supporting the cables; and a wire. When the cables are restrained by the supporter, the cables repeatedly come into contact with the supporter, whereby the cable sheath may be abraded and worn, resulting in damage. When the cables inside the cable carrier are restrained by tie members, freedom of movement of the cables in the longitudinal direction is restricted. In such a case, repeated winding and paying out of the cables may cause local slack in the cables when the cables are pulled, resulting in twisting or entanglement.

### PRIOR ART DOCUMENT

### PATENT DOCUMENT

[Patent Document 1] JP 2023-054929 A

[Patent Document 2] JP 2023-054930 A

[Patent Document 3] JP 2023-054932 A

[Patent Document 4] JP H10-330037 A

[Patent Document 5] JP 2001-251748 A

[Patent Document 5] JP 2008-067908 A

### SUMMARY

### PROBLEM TO BE SOLVED BY INVENTION

An object of the present invention is to prevent occurrence of damage and/or breakage of a plurality of cables to be wound onto or paid out from a spool of a rotating gantry due to deviation or entanglement.

### BRIEF DESCRIPTION OF DRAWINGS

FIG. 1 is a plan view illustrating an overall configuration of a particle beam treatment system in the first embodiment.

FIG. 2 is a side view illustrating a rotating gantry in the first embodiment.

FIG. 3 is a side view illustrating a spool of the rotating gantry in the first embodiment.

FIG. 4 is a rear view of the rotating gantry corresponding to the cross-section taken along the line IV-IV of FIG. 3.

FIG. 5 is a perspective view illustrating straightening plates in the first embodiment.

FIG. 6 is a plan view illustrating straightening wires and the straightening plates in the first embodiment.

FIG. 7 is a side view illustrating lanes of brim disks in the first embodiment.

FIG. 8 is a cross-sectional view illustrating a cable group and an outer shell.

FIG. 9 is a cross-sectional view illustrating the outer diameter of the cable group and the inner diameter of the outer shell.

FIG. 10 is a side view illustrating the lanes of the brim disks in the first modification.

FIG. 11 is an enlarged view illustrating a penetration hole of the spool in the second modification.

FIG. 12 is a plan view illustrating the straightening plates and the straightening wires in the third modification.

FIG. 13 is a rear view illustrating the rotating gantry in the second embodiment.

FIG. 14 is a side view illustrating the spool of the rotating gantry in the second embodiment.

FIG. 15 is a side view illustrating the lane of the brim disks in the second embodiment.

FIG. 16 is an enlarged view illustrating the penetration hole of the spool in fourth modification.

FIG. 17 is a rear view illustrating the rotating gantry in the third embodiment.

FIG. 18 is an enlarged view illustrating the penetration hole of the spool in third embodiment.

FIG. 19 is a side view illustrating the lane of the brim disks in the third embodiment.

FIG. 20 is a rear view illustrating the rotating gantry in the fourth embodiment.

FIG. 21 is a side view illustrating the lanes of the brim disks in the fourth embodiment.

FIG. 22 is a side view illustrating the lanes of the brim disks in the fifth modification.

### DETAILED DESCRIPTION

A cable protection apparatus according to one embodiment of the present invention includes: a rotating gantry configured to rotate about a horizontal axis oriented in a horizontal direction; at least one cable group in which a plurality of cables are bundled, each of the plurality of cables having one end connected to the rotating gantry and another end connected to a stationary apparatus; a spool provided on the rotating gantry and configured to wind or pay out the cable group; a cable straightening apparatus provided in a stationary state at a position below the spool and configured to straighten the cable group hanging down from the spool; and a flexible outer shell that surrounds at least a part of the cable group in a longitudinal direction of the cable group, at least a part of the outer shell being slidable along the longitudinal direction of the cable group.

According to embodiments of the present invention, damage and/or breakage of a plurality of cables to be wound onto or paid out from a spool of a rotating gantry due to deviation or entanglement can be prevented from occurring.

### First Embodiment

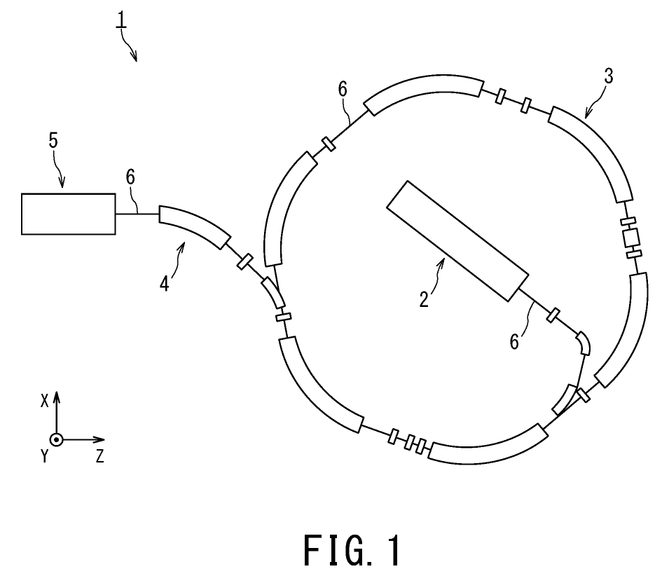

Hereinbelow, embodiments of a cable protection apparatus, a cable protection method, and a particle beam treatment system will be described in detail with reference to the accompanying drawings. First, the first embodiment will be described with reference to FIGS. 1 to 9.

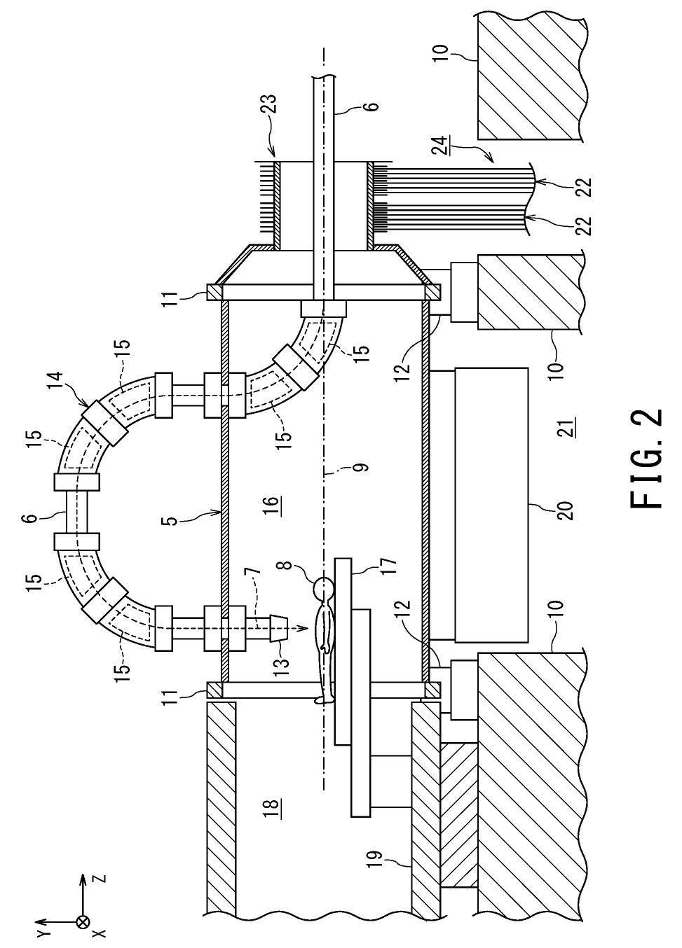

In the following description, the left side of the sheet of each of FIGS. 2, 3, 6, and 7 is assumed to correspond to the front side (i.e., forward side) of a rotating gantry, and the right side of the sheet of each of these figures is assumed to correspond to the rear side (i.e., backward side) of the rotating gantry. In each figure, in an orthogonal coordinate system, the axial direction of the rotating gantry is taken as the Z-axis, the vertical direction (i.e., the up-and-down direction) orthogonal to this Z-axis is taken as the Y-axis, and the horizontal direction orthogonal to both the Z-axis and the Y-axis is taken as the X-axis. The X-axis and Y-axis directions are sometimes referred to as the radial direction of the rotating gantry. The direction of rotation about the axis along the outer circumferential surface of the rotating gantry is sometimes referred to as the circumferential direction.

The reference sign 1 in FIG. 1 denotes the particle beam treatment system according to the first embodiment. The particle beam treatment system 1 performs therapeutic treatment by irradiating a target tissue (e.g., a cancerous lesion) of a patient 8 (see FIG. 2), serving as a subject, with a charged particle beam 7 (see FIG. 2) generated by using charged particles, such as carbon ions. The charged particle beam 7 serves as therapeutic radiation.

A radiation therapy technique using the particle beam treatment system 1 is also referred to as a heavy ion beam cancer treatment technique. This technique is said to be able to damage a cancerous lesion (i.e., focus of disease) and minimize the damage to normal cells by pinpointing the cancerous lesion with carbon ions. The charged particle beams are defined as radioactive rays heavier than electrons, and include proton beams and heavy ion beams, for example. Among these charged particle beams, heavy ion beams are defined as radioactive rays heavier than helium atoms.

As compared with the conventional cancer treatment using X-rays, gamma rays, or proton beams, the cancer treatment using heavy ion beams has the following characteristics: (i) a higher ability to kill the cancerous lesion; (ii) a lower radiation dose at the surface of the body of the patient; and (iii) a radiation dose peaking at the cancerous lesion. Thus, the number of irradiations and side effects can be reduced, and the treatment period can be shortened.

As shown in FIG. 1, the particle beam treatment system 1 includes a beam generator 2, a circular accelerator 3, a beam transport line 4, and a rotating gantry 5.

The beam generator 2 includes an ion source for carbon ions, which are charged particles, and generates a charged particle beam 7 (see FIG. 2) using the carbon ions. The circular accelerator 3 has a ring shape in a plan view and accelerates the charged particle beam 7 generated by the beam generator 2. The beam transport line 4 transports the charged particle beam 7 accelerated by the circular accelerator 3 to the rotating gantry 5. A patient 8 (see FIG. 2) to be irradiated with the charged particle beam 7 is positioned in the rotating gantry 5.

In the particle beam treatment system 1, first, the charged particle beam 7 of carbon ions generated by the beam generator 2 is injected from the beam generator 2 into the circular accelerator 3. The charged particle beam 7 is then accelerated to approximately 70% of the speed of light while circulating around the circular accelerator 3 about one million times. The charged particle beam 7 is then guided to the rotating gantry 5 via the beam transport line 4.

The beam generator 2, the circular accelerator 3, and the beam transport line 4 share a vacuum duct 6 (beam pipe), interior of which is maintained under vacuum. The charged particle beam 7 travels through the interior of the vacuum duct 6. The vacuum duct 6 shared by the beam generator 2, the circular accelerator 3, and the beam transport line 4 forms an integrated structure providing a transport path that guides the charged particle beam 7 to the rotating gantry 5. That is, the vacuum duct 6 is a sealed continuous space having a sufficient degree of vacuum to allow the charged particle beam 7 to pass through.

As shown in the cross-sectional view of FIG. 2, the rotating gantry 5 is an apparatus having a cylindrical shape. The rotating gantry 5 is disposed such that the axis of its cylindrical body is oriented in the horizontal direction. The rotating gantry 5 is rotatable about this horizontal axis 9.

The rotating gantry 5 is supported by a structure 10 of a building constituting a treatment facility in which the particle beam treatment system 1 is installed. For example, end rings 11 are fixed to the front and rear portions of the main body of the rotating gantry 5. Below these end rings 11, rotary drive units 12 are provided. The rotary drive units 12 rotatably support the end rings 11, include drive motors, and are supported by the structure 10. The driving force of the rotary drive units 12 is transmitted to the rotating gantry 5 via the end rings 11, thereby rotating the rotating gantry 5 about the horizontal axis 9.

The rotating gantry 5 is provided with the vacuum duct 6 extending from the beam transport line 4 (see FIG. 1). The vacuum duct 6 is first guided from the rear side of the rotating gantry 5 into the inside along the horizontal axis 9. The vacuum duct 6 once extends outward from the outer circumferential surface of the rotating gantry 5, and then again extends toward the inside of the rotating gantry 5. The tip portion of the vacuum duct 6 extends to a position close to the patient 8.

Of the vacuum duct 6, the portion along the horizontal axis 9 of the rotating gantry 5 is provided with a predetermined rotation mechanism (not shown). Of the vacuum duct 6, the portion outside this rotating mechanism is stationary, and the portion inside this rotating mechanism rotates integrally with the rotating gantry 5.

The rotating gantry 5 is also provided with an irradiation nozzle 13 for irradiating the patient 8 with the charged particle beam 7 and a transport apparatus 14 for transporting the charged particle beam 7 to the irradiation nozzle 13. That is, the irradiation nozzle 13 and the transport apparatus 14 are supported by the rotating gantry 5.

The transport apparatus 14 includes superconducting electromagnets 15 that generate magnetic fields forming a path for transporting the charged particle beam 7. These superconducting electromagnets 15 are, for example, bending magnets for changing the traveling direction of the charged particle beam 7 along the vacuum duct 6 or quadrupole magnets for controlling the focusing and defocusing of the charged particle beam 7.

The irradiation nozzle 13 is provided at the distal end of the vacuum duct 6 and irradiates the patient 8 with the charged particle beam 7 guided by the transport apparatus 14. The irradiation nozzle 13 is fixed to the inner circumferential surface of the rotating gantry 5. The charged particle beam 7 is emitted from the irradiation nozzle 13 in the direction perpendicular to the horizontal axis 9.

Inside the rotating gantry 5, a treatment space 16 for performing particle beam therapy is provided. The patient 8 is placed on a treatment table 17 provided in the treatment space 16. This treatment table 17 can be moved with the patient 8 placed thereon. Positioning can be performed by moving this treatment table 17 such that the patient 8 on this treatment table 17 is moved to the irradiation position of the charged particle beam 7. Thus, the charged particle beam 7 can be delivered to an appropriate site such as the diseased tissue of the patient 8.

The patient 8 is placed at the position of the horizontal axis 9, and the irradiation nozzle 13 can be rotated around the stationary patient 8 by rotating the rotating gantry 5. For example, the irradiation nozzle 13 can be rotated around the patient 8 (i.e., about the horizontal axis 9) clockwise or counterclockwise in increments of 180° when viewed from the rear. The charged particle beam 7 can be delivered from any direction around the patient 8. In other words, the rotating gantry 5 is an apparatus that can change the irradiation direction of the charged particle beam 7 guided by the beam transport line 4 with respect to the patient 8. Thus, the charged particle beam 7 can be radiated in an appropriate direction toward the lesion site with higher precision while reducing the burden on the patient 8.

The charged particle beam 7 loses its kinetic energy to decrease its velocity when passing through the body of the patient 8, experiences a resistance approximately inversely proportional to the square of the velocity, and abruptly stops when its velocity drops to a certain value. The stopping point of the charged particle beam 7 is referred to as the Bragg peak at which high energy is released. The particle beam treatment system 1 adjusts the charged particle beam such that this Bragg peak is at the position of the lesion tissue (i.e., affected tissue) of the patient 8, and thereby can selectively destroy only the lesion tissue while suppressing damage to normal tissues.

The treatment space 16 provided inside the rotating gantry 5 is formed to be integrated with a treatment room 18 located on the front side of the rotating gantry 5. The treatment table 17 is fixed to a floor 19 of the stationary treatment room 18. In other words, it is configured such that the position of the treatment table 17 does not change regardless of the rotation of the rotating gantry 5 and the irradiation nozzle 13.

Of the outer circumferential surface of the rotating gantry 5, on the opposite side of the portion where the transport apparatus 14 is provided, a counterweight 20 is fixed. This counterweight 20 is provided to balance the transport apparatus 14 about the rotating gantry 5. In other words, the weight of the counterweight 20 is set to correspond to the weight of the transport apparatus 14. A weight pit 21 formed in a concave shape in the structure 10 is provided below the rotating gantry 5 in such a manner that the counterweight 20 can pass through the weight pit 21 along with the rotation of the rotating gantry 5.

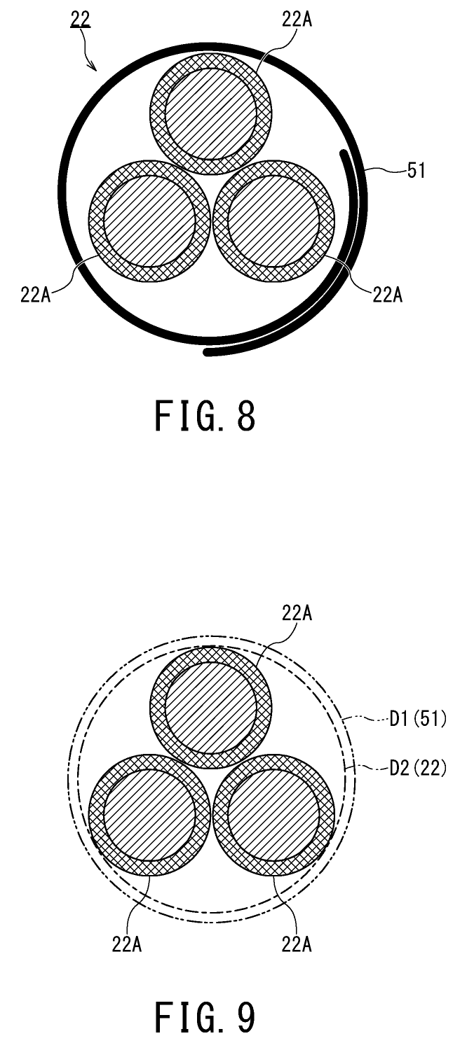

A plurality of cable groups 22 are routed from the outside to the rotating gantry 5. Each cable group 22 is a group of a plurality of cables 22A (see FIG. 8) bundled together. The cables 22A include power supply cables, signal lines, and flexible coolant hoses, for example. The cables 22A are provided to supply power and transmit control signals to specific devices installed in the rotating gantry 5. The cables 22A include flexible hoses that supply a coolant to the superconducting electromagnets 15 included in the transport apparatus 14.

At the rear portion of the rotating gantry 5, a spool 23 is provided. The spool 23 winds or pay outs the cable groups 22 during rotation of the rotating gantry 5. The axis of the spool 23 coincides with the horizontal axis 9 of the rotating gantry 5.

Below the spool 23, a cable pit 24 formed concavely in the structure 10 is provided. In the cable pit 24, the cable groups 22 hanging down from the spool 23 can be disposed. The width dimension of the cable pit 24 in the X-axis direction is set to be greater than the diameter of the spool 23.

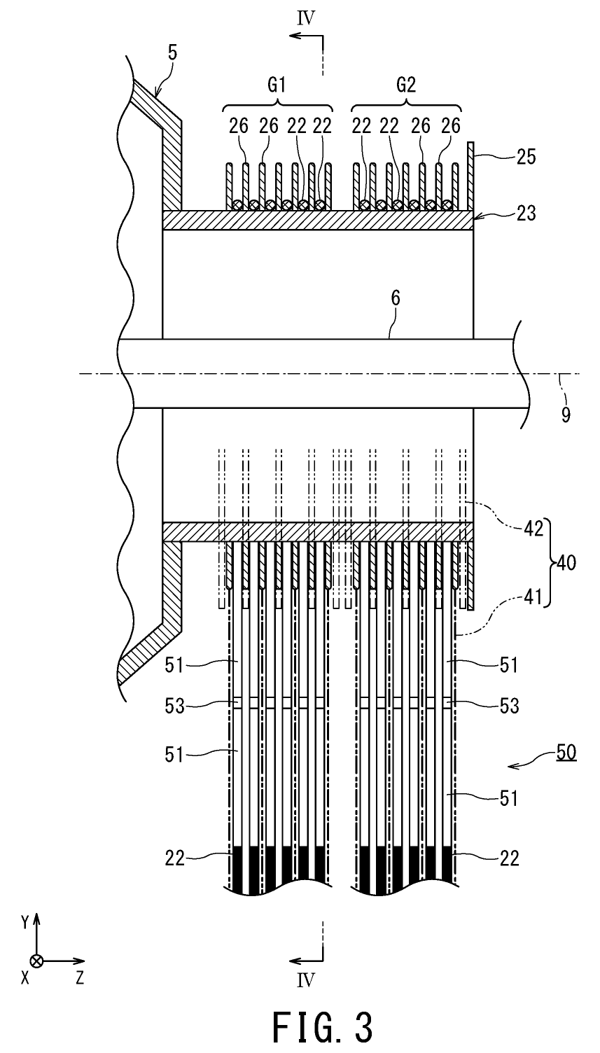

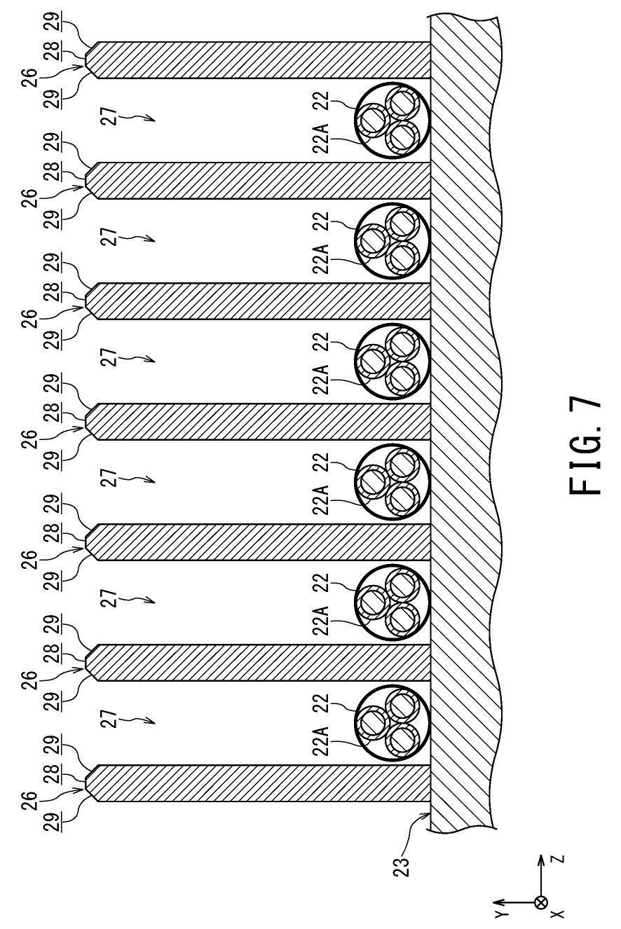

As shown in the cross-sectional view of FIG. 3, the spool 23 is provided to protrude rearward from the rear portion of the rotating gantry 5. The spool 23 is a cylindrical portion and is formed to have a smaller diameter than the diameter of the main body of the rotating gantry 5. The spool 23 includes one disk-shaped flange 25, a plurality of disk-shaped brim disks 26, and a plurality of concave lanes 27 (see FIG. 7) that hold the cable groups 22.

The flange 25 is provided at the rear end portion of the spool 23. The plurality of brim disks 26 are arranged side by side in the axial direction (i.e., in the Z-axis direction) between the flange 25 and the rotating gantry 5. Each brim disk 26 is formed to have a smaller diameter than that of the flange 25. The rear brim disks 26 closest to the flange 25 are positioned at a distance from the flange 25. The plurality of lanes 27 (see FIG. 7) are formed between the respective brim disks 26.

As shown in the cross-sectional view of FIG. 7, each lane 27 accommodates a cable group 22. For example, one lane 27 accommodates one cable group 22. Additionally or alternatively, it may be configured such that one lane 27 accommodates two or more cable groups 22.

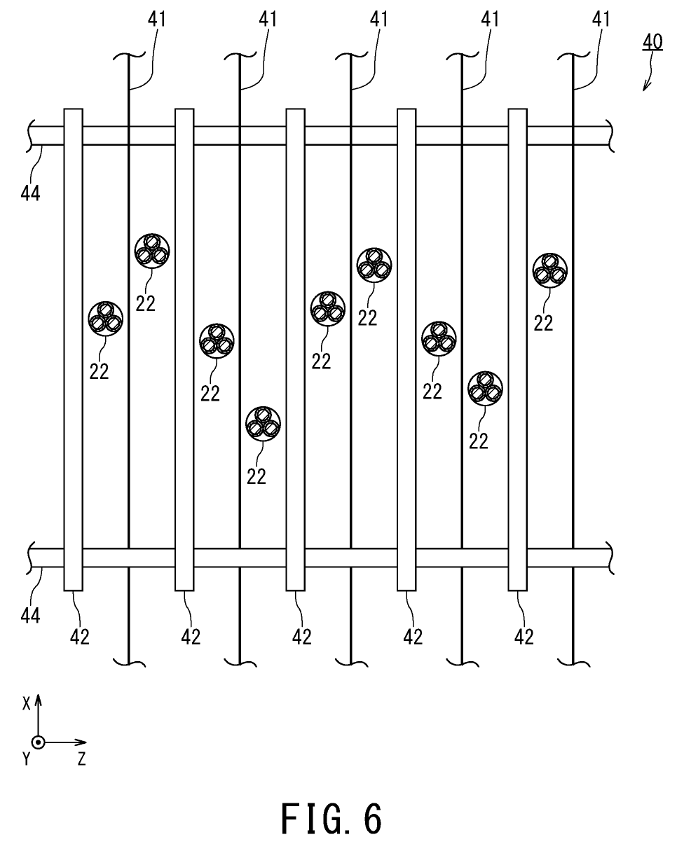

When the cable groups 22 are wound around the spool 23 in the circumferential direction of the spool 23, the cable groups 22 are positioned in line in the axial direction (i.e., in the Z-axis direction) of the spool 23. When the cables 22A hang down from the spool 23, the cable groups 22 are positioned in line in the axial direction (i.e., in the Z-axis direction) as shown in FIG. 6.

The width of each lane 27 may vary depending on the thickness (diameter) or number of the cable groups 22 to be accommodated. A plurality of cable groups 22 of different types or different thicknesses may be accommodated in each lane 27.

On a circumferential surface 28 of each brim disk 26, both corners are cut away to form chamfered portions 29 (i.e., bevels 29). In other words, the chamfered portions 29 are formed around the peripheral edges of the brim disks 26. With this configuration, when the cable groups 22 are accommodated in the lanes 27, the cable groups 22 are less likely to be caught on the brim disks 26. Thus, the friction or tension on the cable groups 22 caused by being caught on the brim disks 26 can be reduced, thereby suppressing irregular winding.

For example, the chamfered portions 29 are inclined surfaces that form an angle of approximately 45° with respect to the protruding direction of the brim disks 26. The provision of the chamfered portions 29 widens the inlet width of each lane 27, thereby allowing the cable groups 22 to be smoothly accommodated in the lanes 27.

Even when the chamfered portions 29 are provided, part of the circumferential surface 28 of each brim disk 26 remains. For example, the circumferential surface 28 of the tip of each brim disk 26 remains. This structure can prevent the cable groups 22 from being cut, being worn out, or being partially abraded even if the cable groups 22 are caught on the brim disk 26.

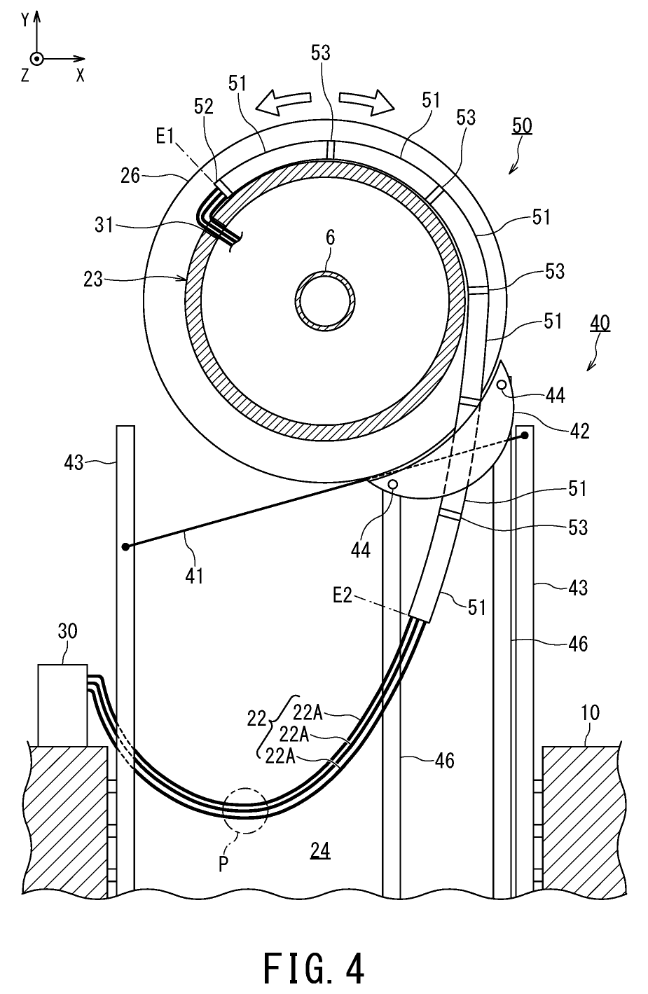

As shown in FIG. 4, each cable group 22 is connected at one end to the spool 23 of the rotating gantry 5, and is connected at the opposite end to a stationary fixing device 30. The fixing device 30 is fixed to the structure 10, for example. The respective cables 22A constituting each cable group 22 are composed of power lines for supplying electric power, signal lines for transmitting control signals, and flexible hoses for supplying the coolant, for example. The fixing device 30 is composed of a power supply, a terminal block, and a coolant supply pump, for example. Although FIG. 4 is a rear view of the rotating gantry 5, for clarity of illustration, respective illustrations of the main body of the rotating gantry 5, the rotary drive units 12, and the transport apparatus 14 are omitted in FIG. 4.

One end of each cable group 22 is introduced into the rotating gantry 5 through a penetration hole 31 formed in the spool 23. The cables 22A are connected to the devices such as the superconducting electromagnets 15 (see FIG. 2) installed in the rotating gantry 5. One end of each cable group 22 is fixed at the penetration hole 31. Each cable group 22 is wound around the spool 23 in the circumferential direction from the fixed penetration hole 31 along the outer periphery of the spool 23.

Each flexible hose is hollow inside and is provided in order to supply the coolant such as liquid helium or liquid nitrogen to the superconducting electromagnets 15. Each flexible hose is configured as a pressure-resistant hose in which metal wires are woven to increase its pressure resistance, and can supply the coolant at a predetermined pressure.

As shown in FIGS. 3 and 4, the plurality of cable groups 22 are divided into a first group G1 and a second group G2. This division of the cable groups 22 into the first and second groups G1 and G2 may be performed on the basis of the type of each cable group 22 or on the basis of the device to which each cable group 22 is connected. In accordance with this, a plurality of brim disks 26 around which the plurality of cable groups 22 of the first group G1 are wound are provided, and another plurality of brim disks 26 around which the plurality of cable groups 22 of the second group G2 are wound are provided.

The cable groups 22 of the first group G1 are different in winding direction around the spool 23 from the cable groups 22 of the second group G2. For example, when the rotating gantry 5 rotates counterclockwise as viewed from the rear, the cable groups 22 of the first group G1 are wound onto the spool 23, whereas the cable groups 22 of the second group G2 are paid out from the spool 23. When the rotating gantry 5 rotates clockwise, the cable groups 22 of the first group G1 are paid out from the spool 23, whereas the cable groups 22 of the second group G2 are wound onto the spool 23.

In FIG. 4, for clarity of illustration, only the cable groups 22 of the first group G1 are illustrated and the cable groups 22 of the second group G2 are omitted. When viewed from the rear, the cable groups 22 of the first group G1 and the cable groups 22 of the second group G2, both hanging down from the spool 23, appear to intersect each other at the cable pit 24.

The particle beam treatment system 1 is provided with cable straightening apparatuses 40 for the rotating gantry 5. In the following, the verb “straighten” is used to refer to disentangling a plurality of cables, untwisting a cable, or making their winding state more regular, in order to prevent these cables from crossing or entangling each other.

Each cable straightening apparatus 40 includes a plurality of straightening wires 41 and a plurality of straightening plates 42. The cable groups 22 pass between the straightening wires 41 and between the straightening plates 42. Each cable straightening apparatus 40 is provided in order to straighten the plurality of cable groups 22 and prevent the cable groups 22 from being irregularly wound.

The cable straightening apparatuses 40 include one for straightening the cable groups 22 of the first group G1 and another for straightening the cable groups 22 of the second group G2. For clarity of illustration, FIG. 4 illustrates only the cable straightening apparatus 40 for the first group G1, and illustration of the cable straightening apparatus 40 for the second group G2 is omitted in FIG. 4. The cable straightening apparatuses 40 for the first group G1 and for the second group G2 have the same configuration and are arranged symmetrically about the rotating gantry 5. For example, when viewed from the rear, the straightening wires 41 of the first group G1 and the straightening wires 41 of the second group G2 appear to cross each other in an X shape.

The plurality of straightening wires 41 are bridged laterally below the spool 23 and are held in a stationary state. These straightening wires 41 are provided to separate the plurality of cable groups 22 hanging down from the spool 23. This structure can prevent the cable groups 22 from being irregularly wound.

The straightening wires 41 are provided at positions corresponding to the respective brim disks 26 and are arranged in the direction in which the brim disks 26 are arranged. With this configuration, the plurality of cable groups 22 arranged in the axial direction can be separated.

For example, in the cable straightening apparatus 40 shown in FIG. 6, the region in which the straightening wires 41 are provided partially or entirely overlaps the region in which the straightening plates 42 are provided. The plurality of straightening wires 41 are stretched so as to be parallel to each other. Each straightening plate 42 is disposed between adjacent straightening wires 41. In a plan view, the straightening plates 42 and the straightening wires 41 are alternately arranged in the axial direction (i.e., in the Z-axis direction). In other words, one straightening wire 41 is stretched between two straightening plates 42, and the straightening wires 41 and the straightening plates 42 are provided parallel to each other.

The plurality of cable groups 22 arranged in the axial direction (i.e., in the Z-axis direction) are separated by at least one of the straightening wires 41 and the straightening plates 42. Each cable group 22 is held in a state of being sandwiched between the straightening wire 41 and the straightening plate 42.

The straightening wires 41 and the straightening plates 42 are arranged in correspondence with the arrangement of the brim disks 26 (see FIG. 3). That is, each cable group 22 disposed between the straightening wire 41 and the straightening plate 42 corresponds to the cable group 22 accommodated in the lane 27. When the cable groups 22 are wound onto or paid out from the spool 23, the cable groups 22 are guided and separated along the straightening wires 41 and the straightening plates 42. Thus, mutual contact between the cable groups 22 is suppressed, friction or tensile load applied to the cable groups 22 due to such contact is reduced, swaying of the cable groups 22 is suppressed, and irregular winding is prevented.

Since the plurality of cable groups 22 are separated by the straightening plates 42, which are rigid members, swaying of the cable groups 22 can be suppressed. The straightening wire 41 as a flexible member contacts the outer peripheral surface of each cable group 22 on a side opposite to the side contacted by the straightening plate 42, so that vibrations of the cable groups 22 can be absorbed.

As shown in FIG. 4, wire mounts 43 extending upward from the bottom surface of the cable pit 24 are fixed to the structure 10 in which the cable pit 24 is formed. For example, a pair of left and right wire mounts 43 spaced apart in the X-axis direction are provided for one straightening wire 41. One end and the other end of each straightening wire 41 are fixed to these wire mounts 43.

The straightening wires 41 are strung in an inclined state with respect to the horizontal direction. With this configuration, the straightening wires 41 come into oblique contact with the cable groups 22 hanging down vertically, thereby reducing the resistance when the straightening wires 41 rub against the cable groups 22. Consequently, wear of the cable groups 22 and occurrence of irregular winding of the cable groups 22 can be reduced.

When the spool 23 is divided into one semicircle on the side where the cable groups 22 hang down and the opposite semicircle, the straightening wires 41 are inclined such that the side from which the cable groups 22 hang down is higher and the opposite side is lower. With this configuration, the angle at which the cable groups 22 come into contact with the straightening wires 41 is reduced, so that the cable groups 22 come into gentle contact with the straightening wires 41.

Each straightening wire 41 is provided at a position adjacent to the brim disk 26 and extends in a tangential direction of the peripheral edge of the brim disk 26. With this configuration, the straightening wires 41 can guide the cable groups 22 at portions where the cable groups 22 are no longer retained by the brim disks 26. Accordingly, friction or tension beyond the expected level of friction or tension does not act on the cable groups 22, and thus, irregular winding can be suppressed.

The plurality of straightening plates 42 are provided in parallel in a stationary state at positions close to the spool 23. These straightening plates 42 are provided below the spool 23 for separating the plurality of cable groups 22 arranged in the axial direction (i.e., in the Z-axis direction). With this configuration, the straightening plates 42 individually separate the cable groups 22 in the axial direction, thereby suppressing an irregular winding state of the cable groups 22.

Each straightening plate 42 is provided at a position corresponding to each brim disk 26, and is arranged in the direction in which the brim disks 26 are arranged. With this configuration, the plurality of cables 22A arranged in the axial direction can be separated.

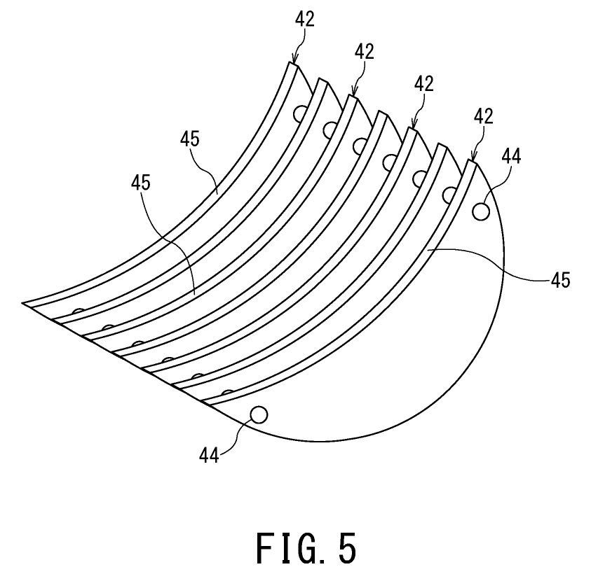

As shown in FIGS. 5 and 6, each straightening plate 42 is a plate-shaped member having a crescent moon shape when viewed from the rear. The straightening plates 42 are connected to each other by connecting members 44 while being spaced apart from each other. The connecting members 44 are rod-shaped members extending in the axial direction (i.e., in the Z-axis direction). These connecting members 44 restrict the horizontal movement range (i.e., movable range in the X-axis direction) of the cable groups 22. Thus, the cable groups 22 hanging down from the spool 23 come to be accommodated within the cable straightening apparatus 40, and consequently, irregular winding does not occur even when the cable groups 22 sway.

Each straightening plate 42 has a curved edge 45 that is curved along the circumferential edge of the brim disk 26. These curved edges 45 are positioned closer to the brim disks 26 than the straightening wires 41. With this configuration, at the position where the cable groups 22 move into and out of the lane 27 (see FIG. 7) between the brim disks 26, the cable groups 22 are guided by the rigid straightening plates 42. Hence, unintended contact between the cable groups 22 is suppressed, swaying of the cable groups 22 is reduced, and occurrence of irregular winding is prevented.

As shown in FIG. 4, plate mounts 46 extending upward from the bottom surface of the cable pit 24 are fixed to the structure 10 in which the cable pit 24 is formed. For example, a plurality of plate mounts 46 spaced apart in the X-axis direction are provided. The connecting members 44 are fixed to these plate mounts 46, and the straightening plates 42 are fixed to the connecting members 44.

Although the straightening plates 42 are provided in a range overlapping with a range in which the straightening wires 41 are provided, other configurations may be adopted. For example, in the axial direction (i.e., in the Z-axis direction), the range in which the straightening wires 41 are provided and the range in which the straightening plates 42 are provided may differ from each other. It may be configured such that some of the cable groups 22 arranged in the axial direction are separated by the straightening plates 42 and the remaining cable groups 22 are separated by the straightening wires 41.

Although the straightening wires 41 are provided at a height position overlapping the height position at which the straightening plates 42 are provided, other configurations may be adopted. For example, the height positions (i.e., positions in the Y-axis direction) at which the straightening plates 42 and the straightening wires 41 are provided may be different from each other. In particular, it may be configured such that the straightening plates 42 are provided in the vicinity of the brim disks 26 and the straightening wires 41 are strung below the straightening plates 42.

Although each cable straightening apparatus 40 includes both the straightening wires 41 and the straightening plates 42, other configurations may be adopted. For example, each cable straightening apparatus 40 may be configured to include either the straightening wires 41 or the straightening plates 42. For example, the cable straightening apparatus 40 may be configured to include only the straightening wires 41 without being provided with the straightening plates 42. Additionally or alternatively, the cable straightening apparatus 40 may be configured to include only the straightening plates 42 without being provided with the straightening wires 41.

The straightening wires 41 or the straightening plates 42 may be provided only at the portions where the cable groups 22 protrude from the spool 23. The cable straightening apparatus 40 may group the cable groups 22 on the basis of their diameters or on the basis of their types. The flexible hoses and the power lines have different bending characteristics, and thus, are grouped on the basis of their type.

Other cables 22A adjacent to one cable group 22 are not required to be bundled. The cable straightening apparatus 40 can separate the cable groups 22 hanging down from the spool 23 from at least one other cable 22A.

Although the plurality of cable groups 22 are arranged in the axial direction (i.e., in the Z-axis direction), other configurations may be adopted. For example, a plurality of cable groups 22 may be arranged side by side in the radial direction of the spool 23 (i.e., in the X-axis and Y-axis directions). A plurality of cable groups 22 may be accommodated in a single lane 27 between the brim disks 26.

Next, a cable protection apparatus 50 provided in the particle beam treatment system 1 will be described. The cable protection method is performed by using this cable protection apparatus 50. As shown in FIGS. 2 to 4, the rotating gantry 5, including the main body and its associated devices and instruments, is also referred to as a rotating gantry system. This rotating gantry system also serves as the cable protection apparatus 50.

The cable protection apparatus 50 includes the rotating gantry 5, at least one cable group 22, the cable straightening apparatuses 40, and at least one outer shell 51.

One cable group 22 is a bundle of a plurality of cables 22A. One outer shell 51 is a flexible member that encloses at least a partial range in the longitudinal direction of one cable group 22 and at least partially slides in the longitudinal direction of the cable group 22.

As shown in FIG. 8, the outer shell 51 is a flexible and tubular (cylindrical) member that accommodates the plurality of cables 22A. The outer shell 51 is a member that restricts the outward degrees of freedom of the bundled cables 22A. The outer shell 51 is formed by wrapping a rubber or fabric sheet around the bundle of the plurality of cables 22A, for example. In the case of FIG. 8, three cables 22A are accommodated in one outer shell 51, thereby constituting one cable group 22.

As shown in FIG. 9, the inner diameter D1 of the outer shell 51 is greater than the virtual outer diameter D2 of the plurality of cables 22A (cable group 22) bundled together. The difference (gap) between the inner diameter D1 of the outer shell 51 and the outer diameter D2 of the bundle of cables 22A can be set arbitrarily. For example, when three cables 22A are accommodated in the outer shell 51, the inner diameter D1 of the outer shell 51 is set to a size capable of accommodating four or more cables 22A. That is, the outer shell 51 envelops the plurality of cables 22A while maintaining a degree of freedom for sliding along the longitudinal direction of the plurality of cables 22A. With this configuration, the outer shell 51 can slide along the longitudinal direction of the bundle of cables 22A. The number of the cables 22A contained within the outer shell 51 may be determined on the basis of the width of the lane 27.

Silicone oil is applied to the range where the outer shell 51 is provided in the longitudinal direction of the cable group 22. With this configuration, the friction between the outer surface of the cable group 22 and the inner surface of the outer shell 51 can be reduced. A degree of freedom for sliding along the longitudinal direction of the cable group 22 can be provided. That is, the outer shell 51 can slide more easily over the outer surface of the cable group 22. The silicone oil is applied using a silicone spray to at least one of the outer surface of the cable group 22 or the inner surface of the outer shell 51, for example.

As shown in FIG. 4, the cable protection apparatus 50 includes at least one binding member 52 and a plurality of retaining members 53. The binding member 52 and the retaining members 53 are members formed in a string or tape shape. For example, the binding member 52 and the retaining members 53 may be cable ties.

The binding member 52 is a member for securing the end portion of the outer shell 51 on the side connected with the spool 23 to the cable group 22. With this configuration, the position of the outer shell 51 in the longitudinal direction of the cable group 22 can be fixed so as not to change regardless of the rotation of the spool 23. That is, when the spool 23 is repeatedly rotated to wind or pay out the cable group 22, the position of the outer shell 51 is prevented by the binding member 52 from shifting. The binding member 52 may also be a member for adhering the outer shell 51 to the cable group 22 using an adhesive.

The plurality of retaining members 53 are members for binding the outer shell 51 to the cable group 22 at predetermined intervals while allowing the outer shell 51 to slide. With this configuration, the outer shell 51 can slide along the longitudinal direction of the cable group 22 while accommodating the cable group 22. For example, a plurality of outer shells 51 are connected by a plurality of retaining members 53. One long outer shell 51 may be wrapped by a plurality of retaining members 53.

Retaining the outer shell 51 at predetermined intervals by the plurality of retaining members 53 can prevents the cables 22A from protruding outside the outer shell 51.

The end portion of the outer shell 51 opposite to the side connected to the spool 23 is not retained by the retaining members 53. That is, the end portion of the outer shell 51 opposite to the side connected to the spool 23 constitutes a free end.

In the longitudinal direction of the cable group 22, the range where the outer shell 51 is provided corresponds to the range through which the cable straightening apparatus 40 passes. For example, the upper end portion E1 of the range where the outer shell 51 is provided is in the vicinity of the penetration hole 31 of the spool 23, and the lower end portion E2 of the range where the outer shell 51 is provided is a portion located below the cable straightening apparatus 40 when the cable group 22 is wound onto the spool 23. With this configuration, the portion of the cable group 22 to be brought into contact with the cable straightening apparatus 40 in the longitudinal direction can be protected by the outer shell 51.

The lower end portion E2 of the range in the longitudinal direction of the cable group 22 with the outer shell 51 provided is located above the lowest point P of the cable group 22 hanging down. With this configuration, the outer shell 51 is positioned above the lowest portion of the cable group 22, and thus, the sheet forming the outer shell 51 can be prevented from curling due to its own weight.

The plurality of cables 22A are bundled into a single unit by the outer shell 51. Accordingly, when the plurality of cables 22A are wound or paid out by the spool 23, the outer shell 51 can prevent the cables 22A from contacting other members, thereby suppressing damage to the cables 22A.

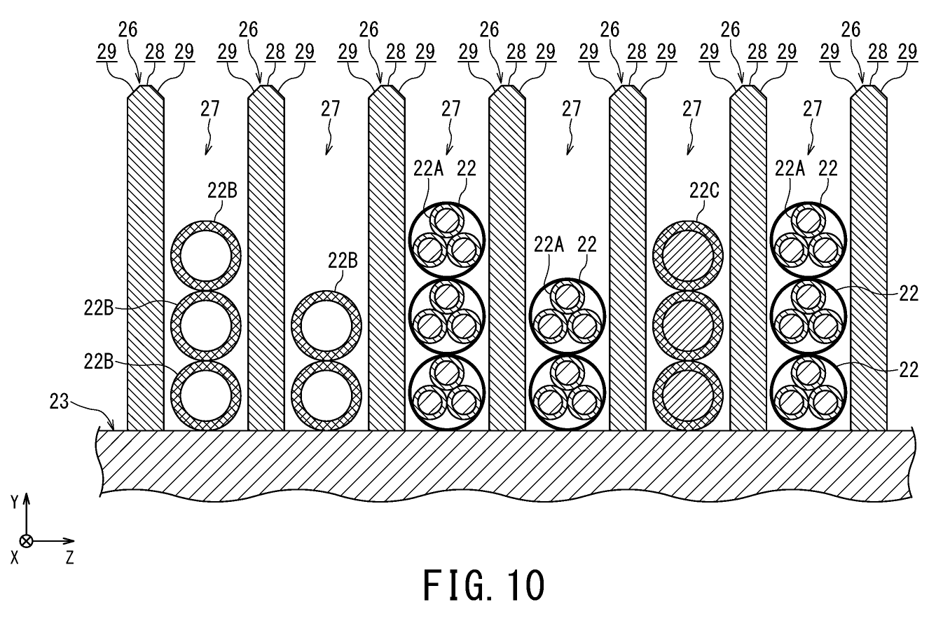

Next, modifications will be described. In the first modification shown in FIG. 10, a plurality of cable groups 22 may be accommodated in each lane 27. Not only the cable groups 22 but also a plurality of unbundled cables 22B and 22C may be accommodated in each lane 27. For example, the cables 22B are flexible hoses, and the cables 22C are power-supply cables.

The brim disks 26 can separate the cable groups 22 from at least one other cable 22B or 22C.

The cables 22A of the cable group 22 and the other cables 22B and 22C may be of different types. For example, they may differ in thickness or stiffness. For each type, the respective cables 22A, 22B, and 22C may have different allowable minimum radii of curvature when being bent.

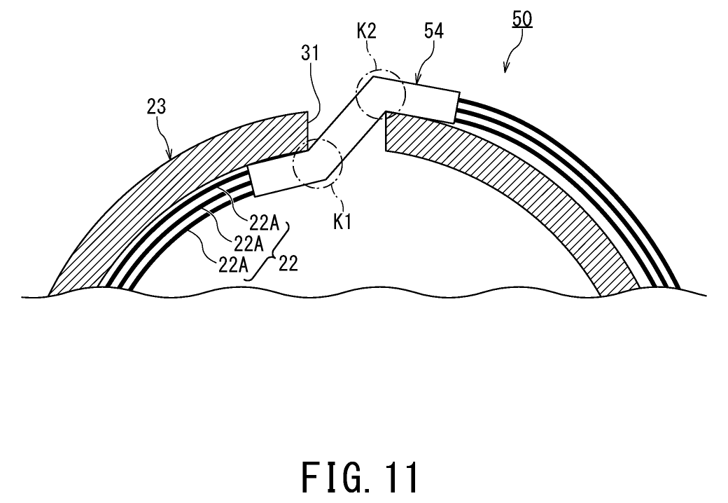

In the second modification shown in FIG. 11, the cable protection apparatus 50 includes a penetration hole 31 and a clamp member 54. In FIG. 11, illustration of other members are omitted to facilitate understanding of the second modification.

The penetration hole 31 is a hole that penetrates the spool 23 and serves to pass the cable group 22 from the outside to the inside of the spool 23. The clamp member 54 is provided in the penetration hole 31, fixed to the outer and inner circumferential surfaces of the spool 23, and is a member for clamping the cable group 22. The penetration hole 31 is formed at a position at which the drawing-out direction or circumferential direction of the cable group 22 to be drawn out from the inside of the spool 23 does not change regardless of rotation of the spool 23. The clamp member 54 is formed as a metal tubular or square pipe, for example. With this configuration, in the longitudinal direction of the cable group 22, the portion subjected to load due to rotation of the spool 23 is fixed by the clamp member 54, thereby preventing damage such as abrasion of the cables 22A.

At least a part of the clamp member 54 is bent. The radius of curvature of each of the bent portions K1 and K2 is greater than the maximum of the allowable minimum radii of curvature of the respective cables 22A included in the cable group 22. With this configuration, bending of the clamp member 54 prevents the cables 22A from being subjected to load. The shape of the clamp member 54 may be straight or curved. However, the clamp member 54 is designed such that the radius of curvature does not fall below the allowable minimum radius of curvature of each cable 22A to be clamped.

Conventionally, when the cables 22A are paid out through the penetration hole 31 of the spool 23, the cables 22A may be pressed against the side surface (i.e., the inner circumferential surface) of the penetration hole 31 by their own weight. At the pressed portion of the penetration hole 31, there occurs an event in which the cables 22A are subjected to a load causing their radii of curvature smaller than their minimum allowable radius of curvature to accelerate their fatigue and thereby leads to failure of the cables 22A earlier than their expected lifetime.

In particular, the cable 22A positioned at the innermost side is especially prone to damage because it is also affected by the other overlapping cables 22A. In response to this event, in the present embodiment, the load applied to the cables 22A at the penetration hole 31 of the spool 23 can be reduced. For example, at the penetration hole 31, the cables 22A are in a clamped state by the clamp member 54. Since the cables 22A are restrained by the clamp member 54, the cables 22A are not affected by external forces and are not damaged significantly earlier than the time point corresponding to their expected lifetime.

In the conventional method of restraining the cables 22A at the inside and outside of the spool 23, the orientation of the cables 22A passing through the penetration hole 31 may become indeterminate depending on the rotational angle of the rotating gantry 5, which may lead to unexpected damage to the cables 22A. The present embodiment can solve this problem.

When the cables 22A are fixed near the penetration hole 31 of the spool 23 by the clamp member 54, the spool 23 is designed so that the rotational angle of the spool 23 keeps the drawing-out direction of the cables 22A always the same.

Adoption of the newly added clamp member 54 near the penetration hole 31 can reduce the load to be applied to the cables 22A in the vicinity of the penetration hole 31 and extend the lifetime of the cables 22A.

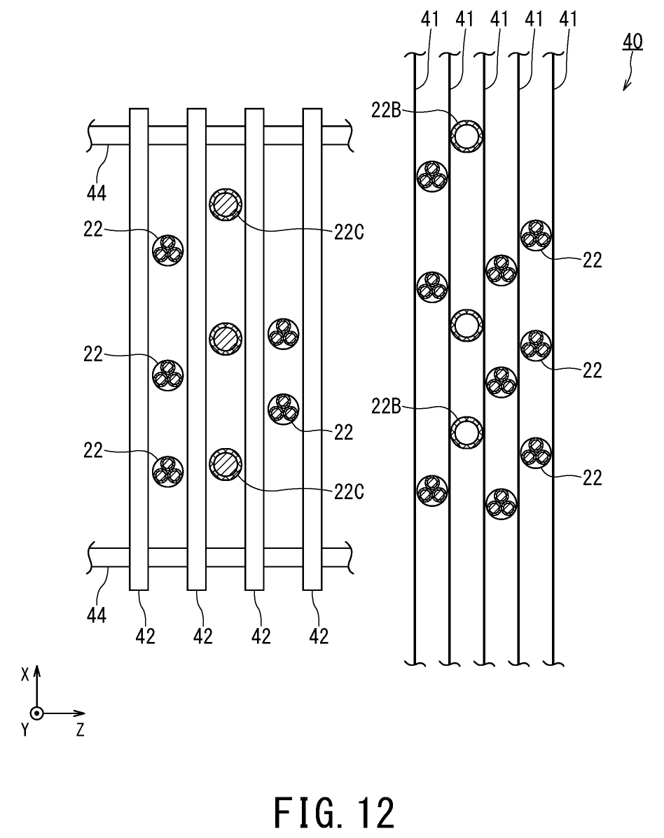

In the cable straightening apparatus 40 of the third modification shown in FIG. 12, the range where the straightening wires 41 are provided and the range where the straightening plates 42 are provided differ from each other in the axial direction (i.e., in the Z-axis direction). For example, a plurality of straightening plates 42 are arranged in a predetermined range along the axial direction, while the straightening wires 41 are arranged in a different range. One or some of the cable groups 22 arranged in the axial direction and the plurality of cables 22B are separated by the straightening wires 41, whereas the plurality of cable groups 22 and the plurality of cables 22C are separated by the straightening plates 42.

For example, the cable groups 22 and the cables 22B and 22C can be separated using members suitable for each type. For example, the cable groups 22 and cables 22B and 22C are suitable for separation by flexible members, and are separated by the straightening wires 41. The cable groups 22 and cables 22B and 22C are suitable for separation by rigid members, and are separated by the straightening plates 42.

### Second Embodiment

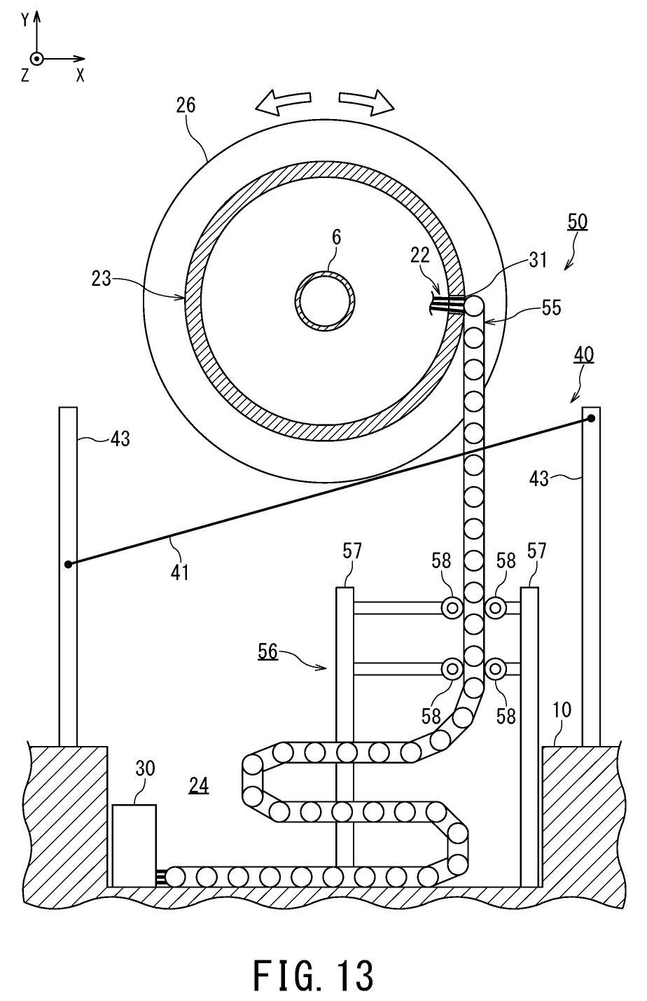

Next, the second embodiment will be described with reference to FIGS. 13 to 15. Components that are the same as those shown in the above-described embodiment are denoted by the same reference numerals, and redundant descriptions are omitted.

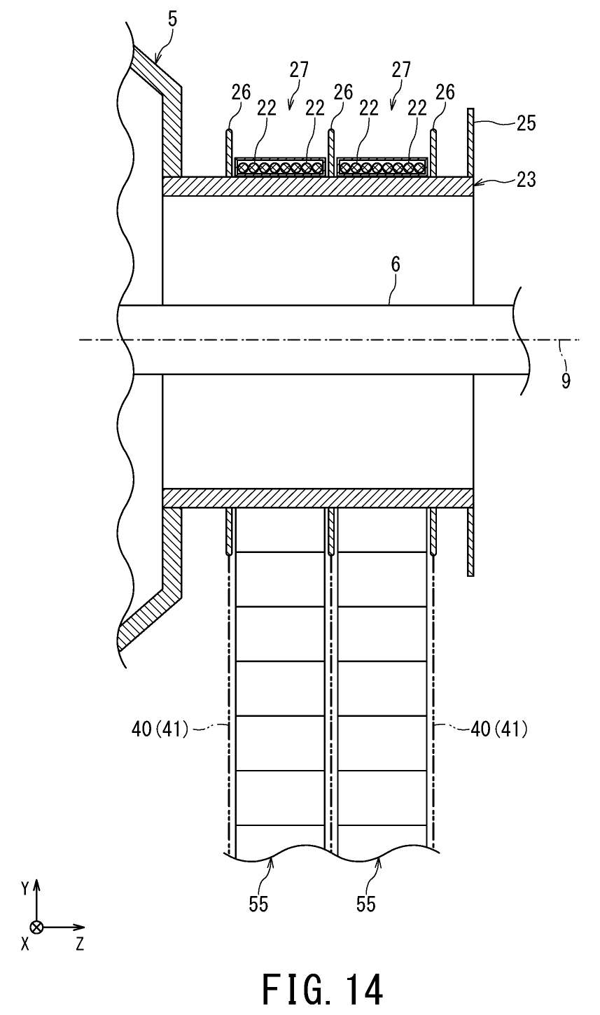

As shown in FIGS. 13 and 14, the cable protection apparatus 50 of the second embodiment includes the penetration hole 31 of the spool 23 and at least one cable carrier 55.

The cable carrier 55 accommodates at least one cable group 22. In the second embodiment, a plurality of cable groups 22 and the cables 22B are accommodated in the cable carrier 55. One end portion of the cable carrier 55 is connected to the portion of the spool 23 corresponding to the penetration hole 31 and extends from the penetration hole 31.

At a lower position of the spool 23, straightening wires 41 serving as the cable straightening apparatus 40 for straightening the cable carrier 55 are provided. The cable pit 24 at the lower position of the spool 23 is provided with a guide apparatus 56 for guiding the cable carrier 55.

The guide apparatus 56 includes a guide support 57 and rollers 58. The guide support 57 is fixed to the bottom surface of the cable pit 24 and extends upward. The plurality of rollers 58 are provided at the upper portion of the guide support 57. The rollers 58 are positioned to straddle the cable carrier 55. When the cable carrier 55 is wound onto or paid out from the spool 23 by rotation of the spool 23, the cable carrier 55 is slidably guided by the rollers 58.

The cable carrier 55 is wound onto and paid out from the spool 23 in the direction perpendicular to the radial direction of the spool 23. In the cable carrier 55 suspended from the spool 23, the guide apparatus 56 is provided between the spool 23 and the bottom of the cable pit 24 surface (i.e., the floor surface).

When the cable carrier 55 is wound onto and paid out from the spool 23, the load on the cable carrier 55 can be minimized by the guide apparatus 56. The guide apparatus 56 may guide the cable carrier 55 at a plurality of locations.

The cable carrier 55 paid out from the spool 23 is accommodated in the cable pit 24. When being wound onto the spool 23, the cable carrier 55 is accommodated in the lane 27 of the brim disk 26.

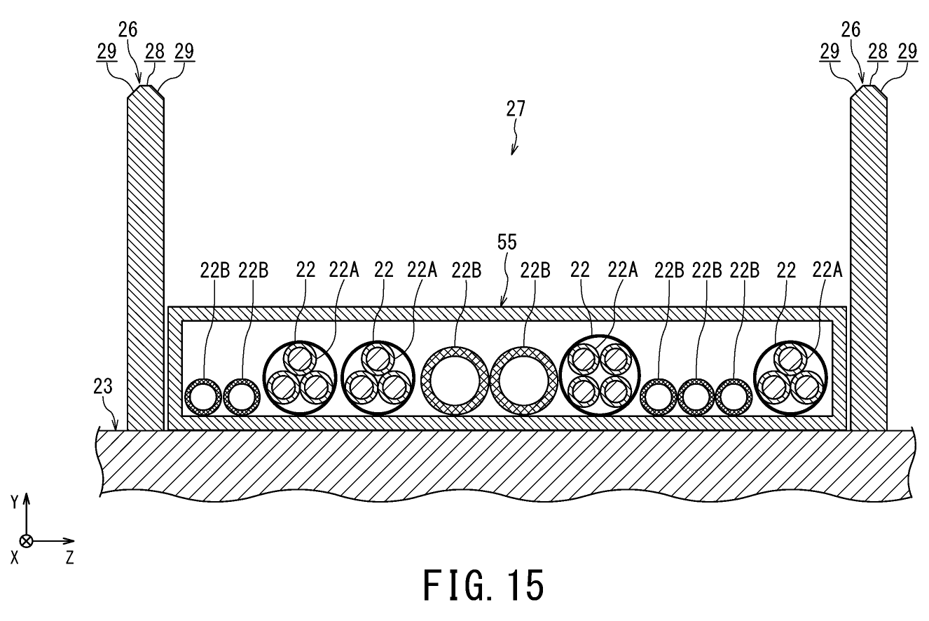

As shown in FIG. 15, the cable groups 22 enclosed by the outer shell 51 are accommodated in the cable carrier 55. Other cables 22B not enclosed by the outer shell 51 are also accommodated in the cable carrier 55.

The plurality of cables 22A accommodated in the cable carrier 55 may include cables having different diameters or stiffnesses. The cable carrier 55 may be disposed by being stacked in the circumferential direction of the spool 23. A plurality of cable carriers 55 may be disposed in parallel with each other in the axial direction of the spool 23. When the cable carriers 55 are arranged in the axial direction, the brim disks 26 between the cable carriers 55 may be omitted.

As shown in FIG. 13, the cable carrier 55 is of a length sufficient to be disposed on the bottom surface of the cable pit 24 (i.e., the floor surface) when being paid out from the spool 23. With this configuration, when the cable carrier 55 is paid out from the spool 23, the load applied to the cable groups 22 due to their own weight can be reduced. Even in a state where the cable carrier 55 is maximally wound onto the spool 23, it is preferred that the lower end of the cable carrier 55 is disposed on the bottom surface of the cable pit 24 (i.e., the floor surface). With this configuration, a portion of the weight of the cable carrier 55 is always applied to the bottom surface of the cable pit 24, thereby reducing the load applied to the cable groups 22.

In the second embodiment, in addition to protection by the outer shell 51, the cable groups 22 can also be protected by the cable carrier 55.

Adoption of the cable carrier 55 and the outer shell 51 covering the cable groups 22 can protect the cables 22A and thereby prevent or suppress damage such as conductor breaks of the cables 22A. Entanglement among the cables 22A in the same lane 27 and consequent deviation from the lane 27 can be prevented in advance of their occurrence. With this configuration, damage to the cables 22A can be prevented or suppressed, and an irregular winding state involving other lanes 27 can also be prevented or suppressed. Consequently, the likelihood of malfunctions is reduced, thereby suppressing a situation where a trouble occurs and treatment is interrupted.

The cable carrier 55 eliminates the need to separate the spool 23 into the lanes 27 or allows the number of the lanes 27 to be reduced. Accordingly, the material and processing costs for forming the lanes 27 can be reduced.

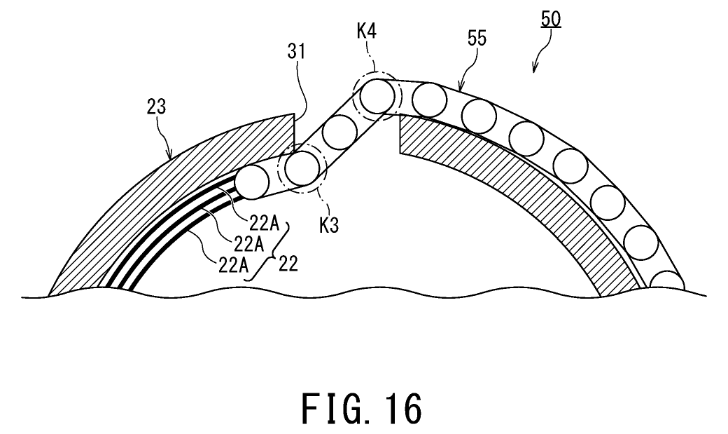

Next, a modification will be described. In the fourth modification shown in FIG. 16, a portion of the cable carrier 55 passes through the penetration hole 31 and is connected to both the outer and inner circumferential surfaces of the spool 23. In FIG. 16, illustration of other members are omitted to facilitate understanding of the fourth modification.

For example, a portion of the cable carrier 55 is disposed inside the spool 23. The penetration hole 31 is formed at a position at which the drawing-out direction of the cable carrier 55 to be drawn out from the inside of the spool 23 (i.e., circumferential direction) does not change regardless of rotation of the spool 23.

At least a portion of the cable carrier 55 is bent. In particular, the cable carrier 55 is bent at the portion of the penetration hole 31. The radii of curvature of the bent portions K3 and K4 are greater than the maximum of the allowable minimum radii of curvature of the respective cables 22A included in the cable group 22. With this configuration, the bending of the cable carrier 55 can prevent a load from being applied to the cables 22A. For example, the bending angles between the respective parts constituting the cable carrier 55 are limited and their radii of curvature are designed so as not to fall below the minimum radii of curvature of the cables 22A to be secured.

### Third Embodiment

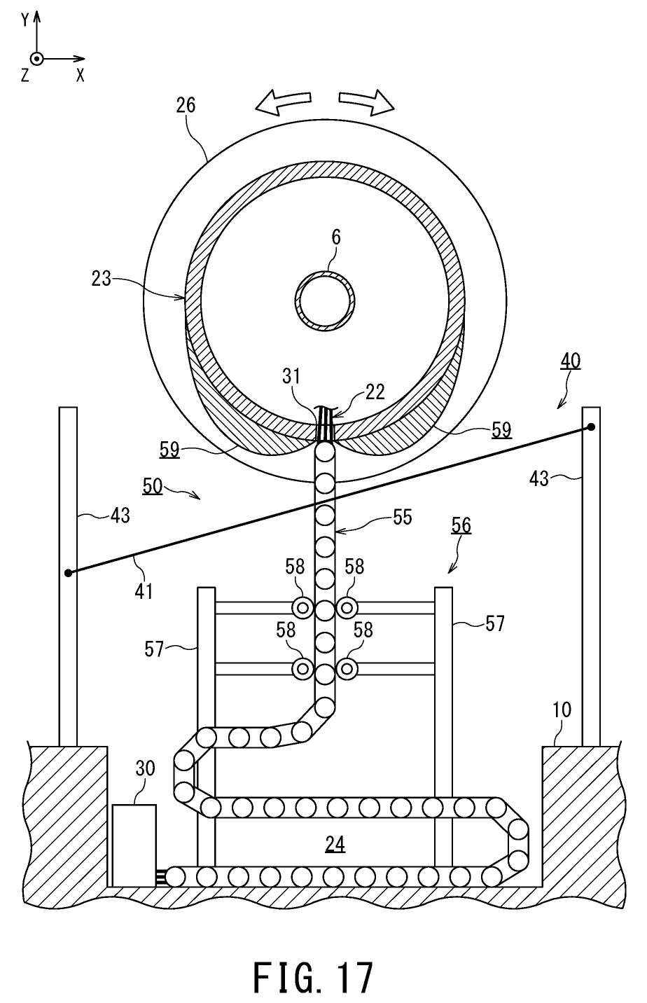

Next, the third embodiment will be described with reference to FIGS. 17 to 19. Components that are the same as those shown in the above-described embodiments are denoted by the same reference numerals, and redundant descriptions are omitted.

As shown in FIG. 17, the cable protection apparatus 50 of the third embodiment includes the penetration hole 31 of the spool 23 and at least one cable carrier 55.

The cable group 22 passes through the penetration hole 31 in the radial direction of the spool 23. The end portion of the cable carrier 55 is connected to the portion of the penetration hole 31 in the outer circumferential surface of the spool 23. When the penetration hole 31 is brought to the lowermost position of the spool 23 by rotation of the spool 23, the cable carrier 55 hangs vertically downward from the penetration hole 31. With this configuration, during maintenance, it becomes easier for maintenance personnel to access the entire range of the cable groups 22 and the cable carrier 55, thereby improving maintainability, safety, and work efficiency.

The cable carrier 55 is based on a reference state in which the penetration hole 31 is at the lowermost position of the spool 23 and the rotation angle is zero degrees, and the cable carrier 55 is configured to be wound in one circumferential direction or the other circumferential direction of the spool 23.

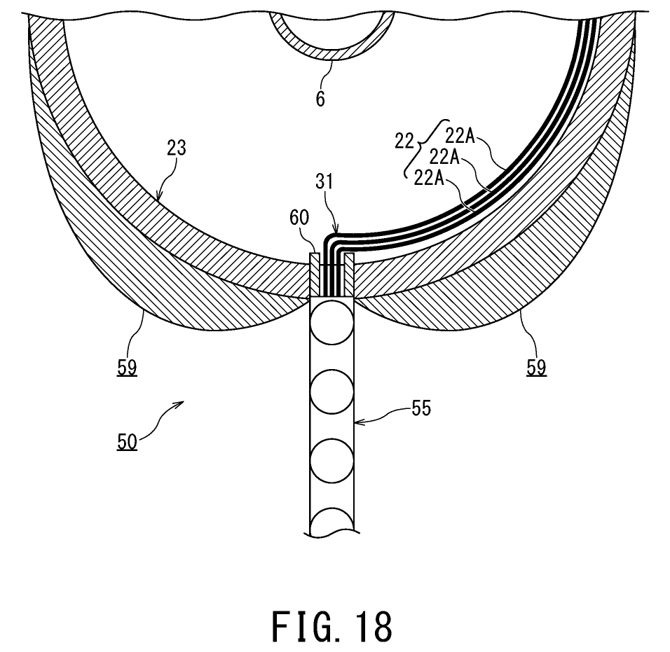

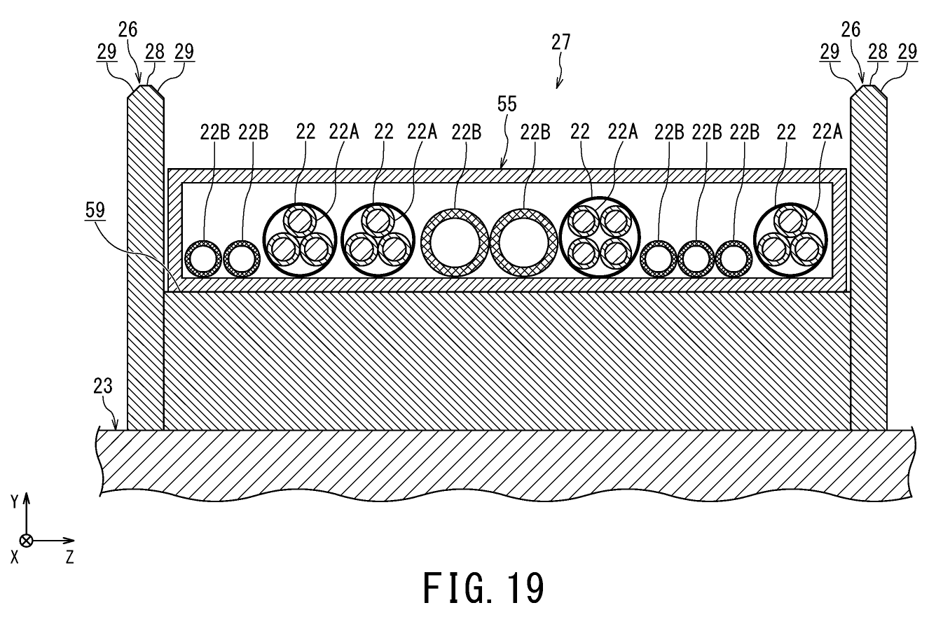

As shown in FIGS. 18 and 19, the portion adjacent to the penetration hole 31 on the outer circumferential surface of the spool 23 is curved to form a guide surface 59 that bulges in the radial direction of the spool 23. With this configuration, when the cable carrier 55 is wound around the spool 23, application of a load to the cables 22B and the cable groups 22 (cables 22A) bent at the portion of the penetration hole 31 can be prevented.

The guide surface 59 is formed by attaching a separate member to a portion of the outer circumferential surface of the spool 23. The outer circumferential surface of the spool 23 itself may be formed as the guide surface 59.

The radius of curvature of the guide surface 59 is greater than the maximum of the allowable minimum radii of curvature of the respective cables 22A included in the cable group 22. With this configuration, application of a load to the cables 22A can be prevented by the guide surface 59.

Provision of the guide surface 59 prevents the cables 22A from falling below their allowable minimum radii of curvature during rotation of the spool 23. Even in the vicinity of the zero-degree position, changes in the radii of curvature of the cables 22A are limited to an amount not exceeding the change in the radii of curvature when the cables 22A are hanging downward.

In the operation of the guide apparatus 56, the connection point of the cable carrier 55 to the spool 23 may move horizontally relative to the floor surface in response to rotation of the spool 23. Accordingly, the guide apparatus 56 may be configured to be movable in the horizontal direction.

In the third embodiment, the cable protection apparatus 50 includes a clamp member 60 that is provided at the penetration hole 31 and circumferentially clamps the cable group 22 passing through the penetration hole 31 in the radial direction of the spool 23. With this configuration, regardless of whether the spool 23 rotates in one circumferential direction or the other circumferential direction, a load applied from the clamp member 60 to the plurality of bundled cables 22A becomes uniform, thereby suppressing wear of the cables 22A.

### Fourth Embodiment

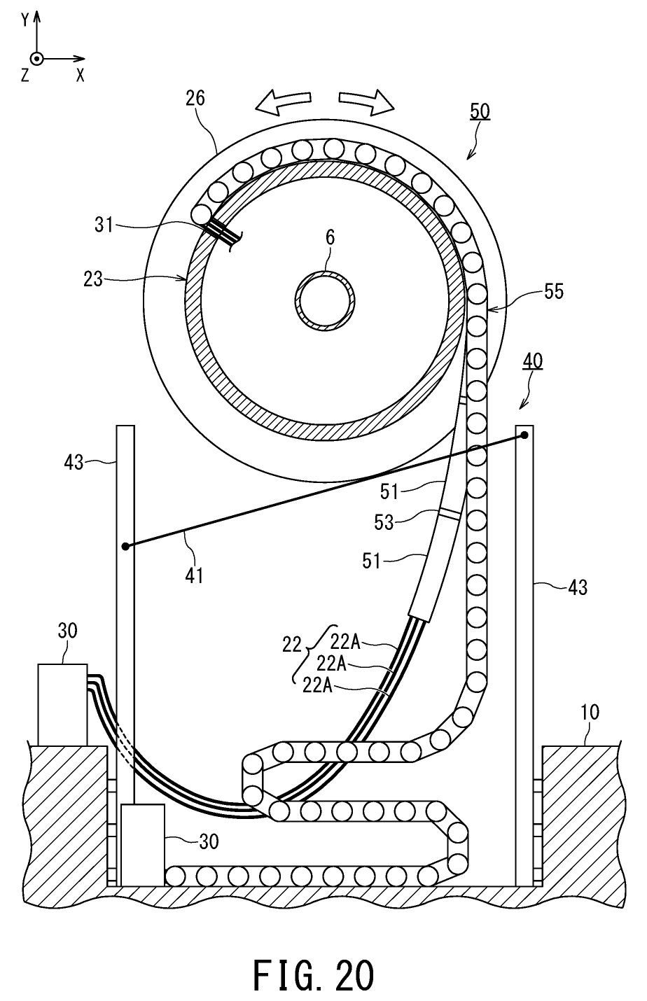

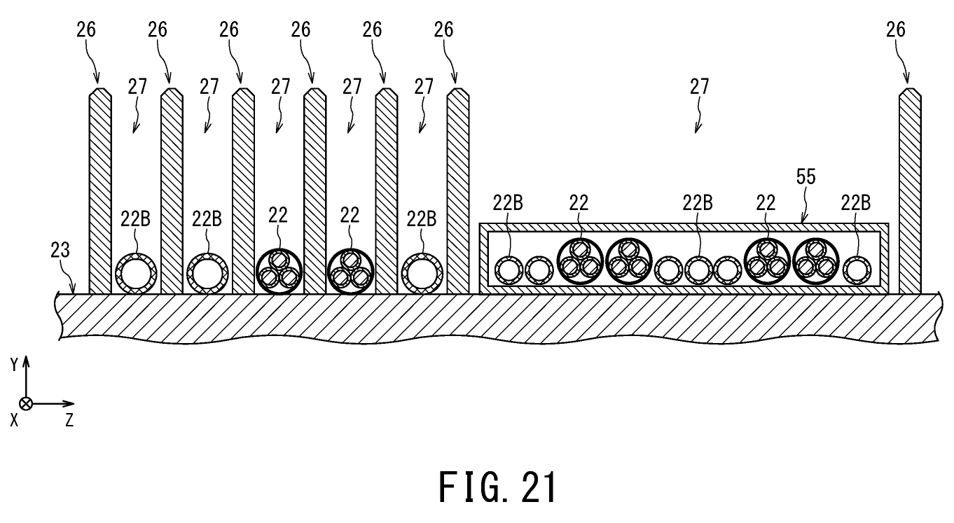

Next, the fourth embodiment will be described with reference to FIGS. 20 and 21. Components that are the same as those shown in the above-described embodiments are denoted by the same reference numerals, and redundant descriptions are omitted.

In the cable protection apparatus 50 of the fourth embodiment shown in FIGS. 20 and 21, the cable groups 22 (cables 22A) covered with the outer shell 51, the cable 22B not covered with the outer shell 51, and the cable carrier 55 are provided in parallel. The cable groups 22 can be protected using both the outer shell 51 and the cable carrier 55

In the fourth embodiment, the plurality of cable groups 22 are protected in manners suitable for their respective types. For example, the plurality of cable groups 22 may be protected by the outer shell 51 or may be protected by the cable carrier 55.

For example, each cable group 22 (cables 22A) having a small diameter and capable of being bundled and enclosed by the outer shell 51 is protected by the outer shell 51. In contrast, thick cables 22B and 22C, such as a power supply cable and a flexible hose, are protected by the cable carrier 55. With this configuration, all of the cable groups 22 (cables 22A) and the cables 22B and 22C in the particle beam treatment system 1 can be protected.

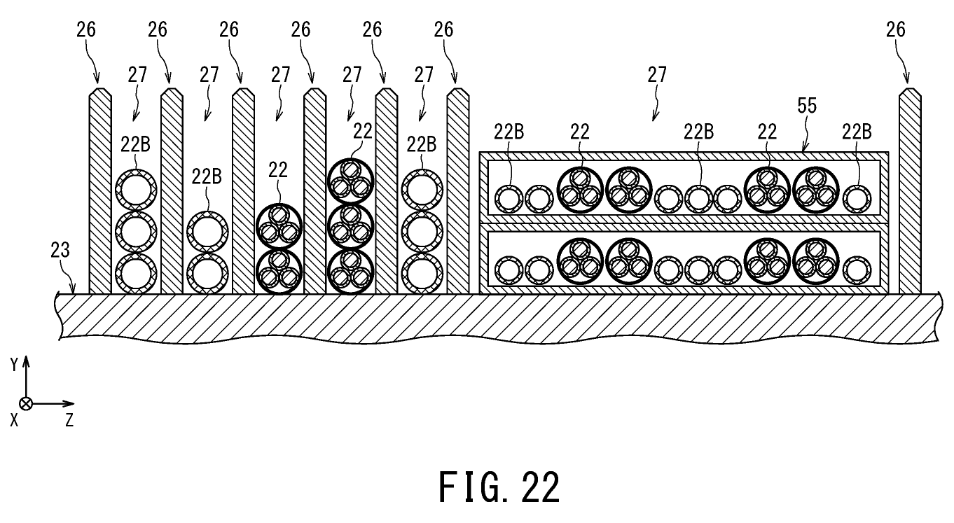

Next, a modification will be described. In the fifth modification shown in FIG. 22, a plurality of cable groups 22 may be accommodated in a single lane 27. Additionally or alternatively, a plurality of cables 22B may be accommodated in a single lane 27. Similarly, a plurality of cable carriers 55 may be accommodated in a single lane 27.

Stacking and winding the plurality of cable groups 22 and the plurality of cable carriers 55 enables a greater number of cables 22A to be arranged on the spool 23 at a higher density.

Although the invention has been described on the basis of the first to fourth embodiments and their modifications, other configurations may be adopted. For example, a configuration applied in any one embodiment or modification may be applied to another embodiment or another modification, and configurations applied in each embodiment or modification may be used in combination.

Although a facility configured to perform heavy-ion-beam cancer treatment is exemplified in the above-described embodiments, the above-described embodiments can also be applied to other facilities. For example, the above-described embodiments may be applied to a facility that performs proton-beam cancer treatment.

Although the foregoing embodiments exemplify the human patient 8 as a treatment subject, other configurations may be adopted. For example, an animal such as a dog and a cat may be a treatment subject. The particle beam treatment system 1 may be used when radiation therapy is performed on such an animal.

The cable straightening apparatus 40 may include a straightening unit having a plurality of rotatable bodies that are configured to separate a plurality of cable groups 22 and to collectively form a cylindrical shape with an outer circumferential surface contacting the cable groups 22. This configuration can suppress an irregular winding state of the cable groups 22 while reducing wear of the cable groups 22.

Conventionally, thin cables 22A, such as signal lines and power lines, have been exposed from the lane 27 or inserted between other cables 22A as a result of repeated winding and paying out caused by rotation of the spool 23, thereby being at risk of damage. In the foregoing embodiments, however, covering the plurality of cables 22A with the outer shell 51 can prevent exposure and damage due to movement of the cables 22A in advance of their occurrence.

The particle beam treatment system 1 requires a large number of cables 22A, which vary in diameter and stiffness. Providing a protection mechanism applicable to each cable 22A is difficult in terms of both cost and process. In contrast, the outer shell 51 of the foregoing embodiments is applicable to any of the cables 22A, thereby providing advantages in terms of both cost and process. The outer shell 51 can be easily replaced, which is also advantageous from a maintenance perspective.

According to at least one embodiment described above, the flexible outer shell 51 is provided, and this flexible outer shell 51 covers at least a partial range in the longitudinal direction of the cable group 22 and is at least partially slidable along the longitudinal direction of the cable group 22. This configuration can prevent damage and/or breakage of the cables 22A to be wound onto or paid out from the spool 23 of the rotating gantry 5 due to deviation and/or entanglement of these cables 22A in advance of their occurrence.

While certain embodiments have been described, these embodiments have been presented by way of example only, and are not intended to limit the scope of the inventions. Indeed, the novel embodiments described herein may be embodied in a variety of other forms; furthermore, various omissions, substitutions, changes, and combinations in the form of the embodiments described herein may be made without departing from the spirit of the inventions. The accompanying claims and their equivalents are intended to cover such forms or modifications as would fall within the scope and spirit of the inventions.

Notwithstanding the foregoing, unless otherwise clearly indicated by the context, a singular expression is not intended to exclude a plural form. Conjunctive terms such as “and” and “or” are inclusive, unless otherwise clearly indicated by the context.

1. A cable protection apparatus comprising:
a rotating gantry configured to rotate about a horizontal axis oriented in a horizontal direction;
at least one cable group in which a plurality of cables are bundled, each of the plurality of cables having one end connected to the rotating gantry and another end connected to a stationary apparatus;
a spool provided on the rotating gantry and configured to wind or pay out the cable group;
a cable straightening apparatus provided in a stationary state at a position below the spool and configured to straighten the cable group hanging down from the spool; and
a flexible outer shell that surrounds at least a part of the cable group in a longitudinal direction of the cable group, at least a part of the outer shell being slidable along the longitudinal direction of the cable group.

2. The cable protection apparatus according to claim 1, wherein a range in which the outer shell is provided in the longitudinal direction of the cable group is at least one of:
(a) a range corresponding to a portion of the cable group passing through the cable straightening apparatus; and
(b) a range with a lower end located at a position higher than a lowest portion of the cable group hanging down.

3. The cable protection apparatus according to claim 1, wherein silicone oil is applied to a range in which the outer shell is provided in the longitudinal direction of the cable group.

4. The cable protection apparatus according to claim 1, further comprising a binding member configured to bind an end portion of the outer shell on a side of the spool to the cable group.

5. The cable protection apparatus according to claim 1, further comprising:
a penetration hole extending through the spool and configured to allow the cable group to pass from an outside of the spool to an inside of the spool; and
a clamp member provided in the penetration hole and fixed to both an outer peripheral surface and an inner peripheral surface of the spool, the clamp member being configured to clamp the cable group.

6. The cable protection apparatus according to claim 5, wherein:
at least a portion of the clamp member is bent; and
a radius of curvature of the bent portion of the clamp member is greater than a maximum of minimum allowable radii of curvature of respective cables included in the cable group.

7. The cable protection apparatus according to claim 1, further comprising a cable carrier configured to accommodate the cable group.

8. The cable protection apparatus according to claim 1, further comprising:
a cable carrier configured to accommodate the cable group; and
a penetration hole extending through the spool and configured to allow the cable group to pass from an outside of the spool to an inside of the spool,
wherein the cable carrier has a length sufficient to be placed on a floor surface when the cable is drawn out from the spool.

9. The cable protection apparatus according to claim 1, further comprising:
a cable carrier configured to accommodate the cable group; and
a penetration hole extending through the spool and configured to allow the cable group to pass from an outside of the spool to an inside of the spool, wherein:
the cable group passes through the penetration hole in a radial direction of the spool;
an end portion of the cable carrier is connected to a portion of the penetration hole at an outer peripheral surface of the spool; and
the cable carrier hangs downward in a vertical direction from the penetration hole when the penetration hole is at a lowermost position of the spool in response to rotation of the spool.

10. The cable protection apparatus according to claim 9, wherein a portion of the penetration hole adjacent to the penetration hole at the outer peripheral surface of the spool forms a guide surface that curves outward in a radial direction of the spool.

11. The cable protection apparatus according to claim 10, wherein a radius of curvature of the guide surface is greater than a maximum of minimum allowable radii of curvature of respective cables included in the cable group.

12. The cable protection apparatus according to claim 9, further comprising a clamp member that is provided in the penetration hole and is configured to clamp the cable group in a circumferential direction, the cable group passing through the penetration hole in a radial direction of the spool.

13. The cable protection apparatus according to claim 9, wherein the cable carrier is a mechanism based on a reference state and is configured to be wound in either one circumferential direction or another circumferential direction around the spool, the reference state being a state in which the penetration hole is at the lowermost position of the spool.

14. A cable protection method using:
a rotating gantry rotatable about a horizontal axis oriented in a horizontal direction;
at least one cable group in which a plurality of cables are bundled, each of the plurality of cables having one end connected to the rotating gantry and another end connected to a stationary apparatus;
a spool provided on the rotating gantry and configured to wind or pay out the cable group;
a cable straightening apparatus provided in a stationary state at a position below the spool and configured to straighten the cable group hanging down from the spool; and
an outer shell surrounding at least a part of a range in a longitudinal direction of the cable group and having flexibility,
the cable protection method comprising a step of causing at least a part of the outer shell to slide in the longitudinal direction of the cable group.

15. A particle beam treatment system comprising:
the cable protection apparatus according to claim 1;
a beam generator configured to generate a charged particle beam;
an accelerator configured to accelerate the charged particle beam;
a transport apparatus supported by the rotating gantry and configured to transport the charged particle beam;
an irradiation nozzle supported by the rotating gantry and configured to irradiate a patient with the charged particle beam that is guided by the transport apparatus in a direction perpendicular to the horizontal axis; and
a treatment table configured to position the patient by moving the patient to an irradiation position of the charged particle beam.

16. The cable protection apparatus according to claim 2, wherein silicone oil is applied to a range in which the outer shell is provided in the longitudinal direction of the cable group.

17. The cable protection apparatus according to claim 2, further comprising a binding member configured to bind an end portion of the outer shell on a side of the spool to the cable group.

18. The cable protection apparatus according to claim 2, further comprising:
a penetration hole extending through the spool and configured to allow the cable group to pass from an outside of the spool to an inside of the spool; and
a clamp member provided in the penetration hole and fixed to both an outer peripheral surface and an inner peripheral surface of the spool, the clamp member being configured to clamp the cable group.

19. The cable protection apparatus according to claim 2, further comprising a cable carrier configured to accommodate the cable group.

20. The cable protection apparatus according to claim 2, further comprising:
a cable carrier configured to accommodate the cable group; and
a penetration hole extending through the spool and configured to allow the cable group to pass from an outside of the spool to an inside of the spool,
wherein the cable carrier has a length sufficient to be placed on a floor surface when the cable is drawn out from the spool.
